(19) United States
(12) Patent Application Publication
(10) Pub. No.: US 2011/0274745 A1
(43) Pub. Date: Nov. 10, 2011

(54) INSTILLATION OF LIPOSOMAL FORMULATION OF SIRNA AND ANTISENSE OLIGONUCLEOTIDES

(75) Inventors: Michael B. Chancellor, Pittsburgh, PA (US); Jonathan Howard Kaufman, Pittsburgh, PA (US)

(73) Assignee: Lipella Pharmaceuticals Inc.

(21) Appl. No.: 12/943,652

(22) Filed: Nov. 10, 2010

Related U.S. Application Data
(60) Provisional application No. 61/259,817, filed on Nov. 10, 2009.

(51) Int. Cl. A61K 9/127; A61P 13/10; A61P 13/02; A61K 31/7088

(52) U.S. Cl. 424/450; 514/44 A

(57) ABSTRACT

A pharmaceutically acceptable deliverable composition and methods for administration of macromolecules for sequence-specific gene-silencing in bladder to treat overactive bladder (OAB), interstitial cystitis/painful bladder syndrome (IC/PBS), lower urinary tract symptoms (LUTS) locally in the bladder, or other diseases or disorders of the bladder or LUTS, has been discovered. In the preferred embodiment, a liposome based delivery system is used to deliver an effective amount of antisense oligonucleotides (ODN) or siRNA that interact with or bind to messenger RNA (mRNA) coding for human nerve growth factor (NGF) to stop the synthesis of NGF.

### CROSS-REFERENCE TO RELATED APPLICATIONS

Priority is claimed to U.S. Provisional Application Ser. No. 61/259,817, filed November 10, 2009.

### FIELD OF THE INVENTION

The present invention generally relates to liposomal delivery of oligonucleotides by distillation into the lumen of an organ to be treated, such as the bladder.

### BACKGROUND OF THE INVENTION

Urinary incontinence, or bladder dysfunction, is loss of bladder control. Symptoms can range from mild leaking to uncontrollable wetting. It can happen to anyone, but it becomes more common with age. Most bladder control problems happen when muscles are too weak or too active. If the muscles that keep the bladder closed are weak, there can be urine leakage when sneezing, laughing or lifting a heavy object. This is stress incontinence. If bladder muscles become too active, there is a strong urge to go to the bathroom when there is little urine in the bladder. This is urge incontinence or overactive bladder. There are other causes of incontinence, such as prostate problems and nerve damage.

Treatment depends on the type of problem. It may include simple exercises, medicines, special devices or procedures prescribed by a doctor, or surgery.

Intravesical therapies have been a mainstay in treatment for many years (Parkin, et al., Urology, 49:105-7 (1997)). Intravesical pharmacotherapy provides high local drug concentrations in the bladder, low risk of systemic side effects and eliminates the problem of low levels of urinary excretion with orally administered agents. A standard instillation time of 30 min has been tested with excellent tolerability in patients. Clinically, dimethylsulfoxide (DMSO) (Rimso-50) is the only FDA approved intravesical treatment for painful bladder syndrome/interstitial cystitis (PBS/IC), believed to have anti-inflammatory properties and mast cell stabilizing effects (Sun and Chai, BJU Int, 90:381-5 (2002)). However success rates of DMSO are generally modest. Bladder epithelium relies primarily on the presence of a surface glycosaminoglycan (GAG) layer and the structural integrity of cell-cell contact, namely tight junctions, to maintain impermeability to toxic urinary wastes (Parsons, et al., Science, 208:605-7 (1980)). When this barrier is damaged, leakage of urine components into the underlying bladder layers initiates the irritative changes in the bladder leading to the stimulation of sensory nerve fibers and the pain, urgency and frequency symptoms(Lavelle, et al., Am J Physiol Renal Physiol., 283:F242-53 (2002)). The urothelium and GAG also presents a significant barrier for effective intravesical drug delivery.

Even more significant is overactive bladder (OAB) which affects 17% of men and women in the United States with increasing incidence with aging, and has an estimated economic burden of $16.4 billion. Many experts believe that 25% with OAB will seek medical therapy. Conservatively, it has been estimated that 25% of those 4 Million OAB patients who seek treatment will fail oral pharmacotherapy and seek therapy for refractory OAB.

In the last several years, several new antimuscarinic medications and improved formulations for OAB have emerged. There were six FDA approved OAB antimuscarinic agents in 2008 with global sales of $2B per year. However, fewer than 20% of patients remain on antimuscarinic therapy due to limited efficacy and adverse effects, such as dry mouth, constipation and cognitive dysfunction. Two procedures often implemented as second-line therapy for antimuscarinic refractory patients are the FDA approved sacral nerve stimulation (SNS) (Interstim, Medtronics Inc.) and intra-detrusor injections of antisense oligonucleotides (ODN) which is in Phase II FDA trials for refractory OAB (Botox, Allergan Inc.,). Several clinical trials are currently being conducted where botulinum toxin is administered. Botulinum toxin has been shown to be helpful to treat refractory overactive bladder (OAB), yet it requires a cystoscopic procedure to directly inject the toxin into the bladder wall. Since the toxin is introduced into the bladder detrusor muscle and can weaken the bladder contractility, up to 43% of patients may develop urinary retention.

The pharmaceutical industry has also shown significant interest in developing therapies for urinary urgency and frequency associated with interstitial cystitis. Most recently, these therapies have included bacillus Calmette-Guerin (BCG), resiniferatoxin (RTX), hyaluronic acid (Cystistat), sodium hyaluronate (SI-7201), and sacral nerve stimulation devices.

Most disease states in lower urinary tract (LUT) infections are driven by overexpression of proteins. Such proteins, either acting directly or through their enzymatic or other functions, contribute in major proportion to many diseases and regulatory functions in man.

Clinical investigations indicate that nerve growth factor (NGF) is an important factor contributing to OAB and detrusor overactivity (DO). NGF is a small secreted protein that influences neuronal development, function, and response to injury (Molliver, et al., Neuron, 19:849-61 (1997)). NGF has been implicated as a chemical mediator of pathology-induced changes in C-fiber afferent nerve excitability linked to mechanical stretch and reflex bladder activity, which can result in DO (De Groat and Yoshimura, Prog Brain Res 152:59-84 (2006); Yoshimura, et al., Prog Neurobiol., 57:583-606 (1999); Yoshimura, et al., J Neurosci., 26, 10847-55 (2006)). Human bladder tissue obtained from subjects undergoing suprapubic prostatectomy for outlet obstruction had more than twice the level of NGF than tissue obtained by cystoscopy from patients who were being evaluated for conditions other than obstruction (Steers, et al., Adv Exp Med Biol., 462:283-92 (1999)); discussion 311-20. Increased levels of urinary NGF have also been detected in patients exhibiting overactive bladder (OAB) symptoms.

Urine NGF levels are increased in patients with neurogenic DO due to spinal cord injury (SCI), idiopathic DO with bladder outlet obstruction (BOO) and sensory urgency (Yokoyama, et al. Neurourol Urodyn, 27:417-20 (2008)). More recently, it was shown that total urine NGF levels were low in controls and in patients with BOO without OAB symptoms, but considerably higher in patients with BOO and OAB symptoms or BOO and DO, indicating the possibility that elevated NGF levels in the bladder might contribute to DO associated with BOO (Liu and Luo, Urology, 72:104-8 (2008)); Neurourol Urodyn, 28:78-81 (2009)). The increase in NGF levels in urine is diagnostic of the bladder as whole instead of just a particular site.

The increased levels of neurotrophic factors including NGF after SCI have been detected in the bladder and in the lumbosacral spinal cord and dorsal root ganglion (DRG) of rats (Seki, et al., J Urol., 168(5):2269-74 (2002); Sasaki, et al., J. Urol., 168(3):1259-64 (2002)). Neutralization of NGF in spinal cord by intrathecal application of NGF antibodies suppressed DO and detrusor-sphincter-dyssynergia in SCI rat also supported the contribution of NGF to lower urinary tract dysfunction after SCI (Seki et al. (2002), Seki, et al., J Urol., 171(1):478-82 (2004)). Recent studies have also shown that NGF can act on several immune cells as well as residential cells (Bonini, et al., Int Arch Allergy Immunol 131:80-4 (2003)). Eosinophils express the functionally active Tyrosine kinase A (TrkA) receptors and NGF activation stimulates eosinophils (Raap, et al., Clin Exp Allergy 38:1493-8 (2008)). Thus it seems likely that NGF not only affects the properties of afferent pathways by direct interactions, but also indirectly influences their properties through induction of inflammatory responses (Adcock, et al., Pulm Pharmacol Ther 22:65-70 (2009); El-Hashim and Jaffal, Thorax 64, 791-7 (2009); Xing, et al., Am J Physiol Heart Circ Physiol 296:H1380-7 (2009)).

For disease states, classical therapeutics have generally focused upon interactions with such proteins to moderate their disease-causing or disease potentiating functions. For example, one pharmaceutical company sponsored testing of systemic anti-NGF therapy using monoclonal human NGF antibodies (tanezumab). The therapeutic benefits from ant-NGF therapy in patients with lower urinary tract (LUT) diseases were attended by side effects such as paresthesia, hypoesthesia and arthralgia.

It is therefore an object of the present invention to provide a composition and method for delivery for inhibiting NGF, especially in the treatment of lower urinary tract symptoms (LUTS) such as OAB, IC/PBS or BOO.

### SUMMARY OF THE INVENTION

A pharmaceutically acceptable deliverable composition and methods for administration of macromolecules for sequence-specific gene-silencing in bladder to treat overactive bladder (OAB), interstitial cystitis/painful bladder syndrome (IC/PBS), lower urinary tract symptoms (LUTS) locally in the bladder, or other diseases or disorders of the bladder or LUTS, has been discovered. In the preferred embodiment, a liposome based delivery system is used to deliver an effective amount of antisense oligonucleotides (ODN) or siRNA that interact with or bind to messenger RNA (mRNA) coding for human nerve growth factor (NGF) to stop the synthesis of NOF.

The preferred intravesical route allows selective exposure of high concentrations of antisense ODN to the NGF producing cells in the urothelium and avoids systemic side effects from genetic manipulation of NGF expression. The intravesical route is also cost effective given the high cost of ODNs such as antisense and siRNA.

### BRIEF DESCRIPTION OF THE DRAWINGS

FIG. 1 illustrates the safety of liposomes on bladder surface compared to 30% ethanol used as a vehicle.

FIG. 2 shows representative cystometrograms CMG of rats following 24 pretreatment with either saline (top), naked ODN (middle trace) or liposomal complex of ODN (bottom trace) by intravesical instillation.

FIGS. 3A, 3B and 3C are summary bar graphs from open cystometry. Bar graphs of ICI (A, min), Amplitude of peak contraction (B, cm H₂O), and pressure threshold of contraction PT (C, cm H₂O) from open cystometry.

FIGS. 4A, 4B and 4C are cystometries of scrambled PNA-TAT (FIG. 4A, top tracing), antisense (FIG. 4C, bottom tracing) and native antisense (FIG. 4B, middle tracing) ODN.

### DETAILED DESCRIPTION OF THE INVENTION

Gene-silencing (for example, antisense or siRNA therapeutics) involves introduction of short strands of DNA with sequences complementary to the mRNA of a particular gene that can bind to the mRNA encoding the encoded protein, preventing the protein from being synthesized by the ribosome. Drug development of this approach has been hampered by inefficient intracellular delivery and cellular uptake of the ODN. Commonly used phosphodiester and phosphorothioate antisense oligonucleotides (ODN) are polyanions and therefore cannot passively diffuse across most cell membranes including the glycoaminoglycan layer (anionic charge) lining the bladder lumen. There has been lot of interest in development of effective delivery agents for realizing the therapeutic potential of ODN. Liposomes resemble cell membranes in their structure and have been used to enhance cellular uptake of ODN.

### I. Compositions

A. Oligonucleotides

As used herein the term “isolated” is meant to describe a compound of interest (e.g., either a polynucleotide or a polypeptide) that is in an environment different from that in which the compound naturally occurs e.g. separated from its natural milieu such as by concentrating a peptide to a concentration at which it is not found in nature. “Isolated” is meant to include compounds that are within samples that are substantially enriched for the compound of interest and/or in which the compound of interest is partially or substantially purified.

As used herein, “isolated nucleic acid” refers to a nucleic acid that is separated from other nucleic acid molecules that are present in a mammalian genome, including nucleic acids that normally flank one or both sides of the nucleic acid in a mammalian genome.

As used herein, “operably linked” means incorporated into a genetic construct so that expression control sequences effectively control expression of a coding sequence of interest.

As used herein, a “fragment” of a polypeptide refers to any subset of the polypeptide that is a shorter polypeptide of the full length protein. Generally, fragments will be five or more amino acids in length.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “polypeptide” refers to a chain of amino acids of any length, regardless of modification (e.g., phosphorylation or glycosylation).

As used herein, an “amino acid sequence alteration” can be, for example, a substitution, a deletion, or an insertion of one or more amino acids.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences

As used herein, an “expression control sequence” is a DNA sequence that controls and regulates the transcription and/or translation of another DNA sequence.

“Operably linked” refers to an arrangement of elements wherein the components so described are configured so as to perform their usual or intended function. Thus, two different polypeptides operably linked together retain their respective biological functions while physically linked together.

As used herein, “valency” refers to the number of binding sites available per molecule.

As used herein, the term “host cell” refers to prokaryotic and eukaryotic cells into which a recombinant expression vector can be introduced.

As used herein, “transformed” and “transfected” encompass the introduction of a nucleic acid (e.g. a vector) into a cell by a number of techniques known in the art.

The terms “individual”, “host”, “subject”, and “patient” are used interchangeably herein, and refer to a mammal, including, but not limited to, humans, rodents such as mice and rats, and other laboratory animals.

The terms “inhibitory ribonucleic acid” or “inhibitory RNA” refer to a RNAs specific for target RNAs that reduce of inhibit the expression of the target RNA. Representative inhibitory nucleic acids include, but are not limited to dsRNA, siRNA, shRNA, miRNA, piRNA, external guide sequences, ribozymes, and other short catalytic RNAs. Expression of the target nucleic acid can be inhibited at the transcriptional or translational level.

Inhibitory RNAs are configured to hybridize to target mRNAs and modulate their expression or integrity. Inhibitory RNAs can modulate target mRNA expression through several means, including directly catalyzing target mRNA degradation, causing the recruitment of cellular proteins and enzymes that mediate mRNA degradation, inhibiting or reducing the translation of target mRNA, or otherwise reducing the stability of target mRNA. Inhibitory RNAs can be single-stranded or double-stranded. Exemplary inhibitory RNAs include, but are not limited to, dsRNA, siRNA, shRNA, miRNA, piRNA, external guide sequences, ribozymes, and other short catalytic RNAs.

Inhibitory RNAs are complementary to their target RNAs. The term “complementary”, as used herein, refers to the capacity of two nucleotides to pair precisely with each other. This term may also be used to refer to oligonucleotides which exhibit the ability of pairing precisely with each other. For example, if the nucleotides located at a certain position on two oligonucleotides are capable of hydrogen bonding, then the oligonucleotides are considered to be complementary to each other at that position. The inhibitory RNAs and the target RNAs are complementary to each other when a sufficient number of corresponding positions in each molecule are occupied by nucleotides which can hydrogen bond with each other. Thus, “complementary” is a term that is used to indicate a sufficient degree of complementarity or precise paring such that stable and specific binding may occur between the inhibitory RNA and the target RNA. It is understood in the art that the sequence of an inhibitory oligonucleotide compound need not be 100 percent complementary to that of its target RNA. A sufficient degree of complementarity prevents non-specific binding of the inhibitory oligonucleotide compound to nontarget sequences under conditions in which specific binding is desired, i.e. under physiological conditions.

Originally, studies gene silencing were performed using ODN of native DNA or RNA. However, they have some problems such as: (1) rapid degradation and inactivation of native ODN due to endogenous nucleases; (2) difficulty for negatively charged oligonucleotides to penetrate through cell membrane into cells due to negatively charged membrane; and the like. Considering such problems, variously modified ODN having the basic structure of DNA or RNA have been developed and utilized. ODN can also be custom synthesized to contain properties that are tailored to fit a desired use and overcome their inherent problems. Among these modified oligonucleotides, phosphorothioate-type oligonucleotides are the most practical ones, wherein diester bonds between respective nucleosides are substituted with phosphorothioate bonds (See generally Uhlmann and Peymann, 1990, Chemical Reviews 90, at pages 545-561 and references cited therein, Padmapriya and Agrawal, 1993, Bioorg. & Med. Chem. Lett. 3,761). While the introduction of sulfur atoms (phosphothioate) to the back bone slows the enzyme degradation rate, it also creates other problems of toxicity, and chirality at the same time. ODN also tend to form secondary and high-order solution structures and once these structures are formed, they become targets of various enzymes, proteins, RNA, and DNA for binding. This results in nonspecific side effects and reduced amounts of active compound binding to mRNA. The longer the compound remains outside the cell, the more degraded it becomes resulting in less active compound arriving at the target. Native ODN were unsuitable for use by intravesical route.

Oligodeoxynucleotides can include or be a phosphorodiester backbone, a phosphorothioate backbone, locked nucleic acid, peptide nucleic acid, tricyclo-DNA, decoy oligonucleotide, ribozymes, spiegelmers (containing L nucleic acids, an apatamer with high binding affinity), or CpG oligomers.

Inhibitory RNAs can contain any known base analogs including, but not limited to, 4-acetylcytosine, 8-hydroxy-N-6-methyladenosine, aziridinylcytosine, pseudoisocytosine, 5-(carboxyhydroxylmethyl) uracil, 5-bromouracil, 5-carboxymethylaminomethyl-2-thiouracil, 5-carboxymethylaminomethyluracil, dihydrouracil, inosine, N6-isopentenyladenine, 1-methyladethne, 1-methylpseudouracil, 1-methyl guanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, 2,6-diaminopurine, and 2′-modified analogs such as, but not limited to 0-methyl, amino-, and fluoro-modified analogs.

### i. siRNA and shRNA

In one embodiment, the inhibitory RNAs are siRNAs or shRNAs. The term “siRNA” means a small interfering RNA that is a short-length double-stranded RNA that is not toxic. Generally, there is no particular limitation in the length of siRNA as long as it does not show toxicity. “siRNAs” can be, for example, 15 to 49 bp, preferably 15 to 35 bp, and more preferably 21 to 30 by long.

Inhibitory nucleic acids and methods of producing them are well known in the art. siRNA design software is available for example at http://i.cs.hku.hk/˜sirna/software/sirna.php. The sequence of at least one strand of the siRNA contains a region complementary to at least a part of the target mRNA sufficient for the siRNA to specifically hybridize to the target mRNA. In one embodiment, the siRNA is substantially identical to at least a portion of the target mRNA. “Identity”, as known in the art, is the relationship between two or more polynucleotide (or polypeptide) sequences, as determined by comparing the sequences. In the art, identity also means the degree of sequence relatedness between polynucleotide sequences, as determined by the match between strings of such sequences. Identity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, N.J., 1994; Sequence Analysis in Molecular Biology, von Heinje, G., Academic Press, 1987; and Sequence Analysis Primer, Gribskov, M. and Devereux, J., eds., M Stockton Press, New York, 1991). While there are a number of methods to measure identity between two polynucleotide sequences, the term is well known to skilled artisans (Sequence Analysis in Molecular Biology, von Heinje, G., Academic Press, 1987; Sequence Analysis Primer, Gribskov, M. and Devereux, J., eds., M Stockton Press, New York, 1991; and Carillo and Lipman, SIAM J. Applied Math., 48:073 (1988)). Methods commonly employed to determine identity between sequences include, but are not limited to those disclosed in Carillo and Lipman, SIAM J. Applied Math., 48:1073 (1988). Preferred methods to determine identity are designed to give the largest match between the sequences tested. Methods to determine identity are codified in computer programs. Computer program methods to determine identity between two sequences include, but are not limited to, GCG program package (Devereux, et al., Nucleic Acids Research, 12(1):387 (1984)), BLASTP, BLASTN, and FASTA (Atschul, et al., J. Molec. Biol., 215:403 (1990)). Another software package well known in the art for carrying out this procedure is the CLUSTAL program. It compares the sequences of two polynucleotides and finds the optimal alignment by inserting spaces in either sequence as appropriate. The identity for an optimal alignment can also be calculated using a software package such as BLASTx. This program aligns the largest stretch of similar sequence and assigns a value to the fit. For any one pattern comparison several regions of similarity may be found, each having a different score. One skilled in the art will appreciate that two polynucleotides of different lengths may be compared over the entire length of the longer fragment. Alternatively small regions may be compared. Normally sequences of the same length are compared for a useful comparison to be made.

In one embodiment, the inhibitory nucleic acid has 100% sequence identity with at least a part of the target mRNA. However, inhibitory nucleic acids having 70%, 80% or greater than 90% or 95% sequence identity may be used. Thus sequence variations that might be expected due to genetic mutation, strain polymorphism, or evolutionary divergence can be tolerated.

The duplex region of the RNA may have a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

The double-stranded RNA portions of siRNAs in which two RNA strands pair up are not limited to the completely paired ones, and may contain nonpairing portions due to mismatch (the corresponding nucleotides are not complementary) or bulge (lacking in the corresponding complementary nucleotide on one strand). Nonpairing portions can be contained to the extent that they do not interfere with siRNA formation. Suitable siRNAs can contain one or more modified bases, or have a modified a backbone to increase stability or for other reasons. For example, the phosphodiester linkages of natural RNA may be modified to include at least one of a nitrogen or sulfur heteroatom. siRNAs comprising unusual bases, including, but not limited to inosine, or modified bases, such as tritylated bases, can be used. The term “siRNA”, as used herein, embraces such chemically, enzymatically or metabolically modified forms of siRNA.

The terminal structure of siRNA may be either blunt or cohesive (overhanging) as long as siRNA can silence, reduce, or inhibit the target gene expression due to its RNAi effect. The cohesive (overhanging) end structure is not limited only to the 3′ overhang, and the 5′ overhanging structure may be included as long as it is capable of inducing the RNAi effect. The number of overhanging nucleotide can be any numbers as long as the overhang is capable of inducing the RNAi effect. For example, the overhang can consist of 1 to 8, or preferably 2 to 4 nucleotides.

The terminal structure of the siRNA is not necessarily the cut off structure at both ends as described above, and may have a stem-loop structure in which ends of one side of double-stranded RNA are connected by a linker RNA. siRNAs containing a linker RNA that forms a hairpin structure are referred to as short hairpin RNAs, or shRNAs. The length of the double-stranded RNA region (stem-loop portion) can be, for example, 15 to 49 bp, preferably 15 to 35 bp, and more preferably 21 to 30 by long. Alternatively, the length of the double-stranded RNA region that is a final transcription product of siRNAs to be expressed is, for example, 15 to 49 bp, preferably 15 to 35 bp, and more preferably 21 to 30 by long. Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion.

### ii. miRNA

Micro RNAs (referred to as “miRNAs”) are small non-coding RNAs, belonging to a class of regulatory molecules found in plants and animals that control gene expression by binding to complementary sites on target messenger RNA (mRNA) transcripts. miRNAs are generated from large RNA precursors (termed pri-miRNAs) that are processed in the nucleus into approximately 70 nucleotide pre-miRNAs, which fold into imperfect stem-loop structures (Lee, et al., Nature, 425(6956):415-9 (2003)). The pre-miRNAs undergo an additional processing step within the cytoplasm where mature miRNAs of 18-25 nucleotides in length are excised from one side of the pre-mlRNA hairpin by an RNase III enzyme, Dicer (Hutvagner, et al., Science, 12:12 (2001) and Grishok, et al., Cell, 106(1):23-34 (2001)). MiRNAs have been shown to regulate gene expression in two ways. First, miRNAs that bind to protein-coding mRNA sequences that are exactly complementary to the miRNA induce the RNA-mediated interference (RNAi) pathway. Messenger RNA targets are cleaved by ribonucleases in the ribonucleoprotein complex known as the RISC complex. This mechanism of miRNA-mediated gene silencing has been observed in plants (Hamilton and Baulcombe, Science, 286(5441):950-2 (1999) and Reinhart, et al., Genes and Dev., 16:1616-1626 (2002)), and in animals (Yekta, et al., Science, 304(5670):594-6 (2004)). In the second mechanism, miRNAs that bind to imperfect complementary sites on messenger RNA transcripts direct gene regulation at the posttranscriptional level but do not cleave their mRNA targets. MiRNAs identified in both plants and animals use this mechanism to exert translational control of their gene targets (Bartel, Cell, 116(2):281-97 (2004)).

Preferred miRNAs have at least 80%, typically at least 90%, even more typically at least 95% sequence identity with the target mRNA. Naturally occurring microRNAs that regulate target RNAs, pri-miRNA, pre-miRNA, mature miRNA or fragments of variants thereof that retain the biological activity of the mature miRNA and DNA encoding a pri-miRNA, pre-miRNA, mature miRNA, fragments or variants thereof, or regulatory elements of the miRNA, have been identified. The size of the miRNA is typically from 21 nucleotides to 170 nucleotides, although nucleotides of up to 2000 nucleotides can be utilized. In a preferred embodiment the size range of the pre-miRNA is between 70 to 170 nucleotides in length and the mature miRNA is between 21 and 25 nucleotides in length.

### iii. piRNA

Recent studies have revealed a new class of 24- to 30-nt RNAs that are generated by a Dicer-independent mechanism and that interact with a subset of Argonaute proteins related to Piwi. Studies in Drosophila have identified five Argonaute proteins: Ago1, Ago2, Ago3, Piwi, and Aubergine (Aub). Ago1 and Ago2 are ubiquitously expressed, whereas the expression of Piwi, Aub, and Ago3 are germ line-specific. Ago1 associates with miRNAs to regulate endogenous gene expression, and Ago2 serves as the slicer for siRNA-mediated gene silencing. Piwi, Aub, and Ago3 have been recently reported to interact with 24- to 30-nt small RNAs known as rasiRNAs. Murine Ago1, Ago2, Ago3, and Ago4 are associated with miRNAs, while the Piwi orthologs, MIWI, MILI, and MIWI2, are found in germ line cells.

Like other members of the Ago family, Piwi proteins associate with small RNAs that act as guides in silencing target mRNA. These Piwi-interacting RNAs are called piRNAs. These small RNAs associated with Piwi RNPs have been cloned and sequence analysis of piRNAs shows a high percentage of uridine residues at the 5′ termini (Gunawardane, et al., Science, 315(5818):1587-90 (2007), and genomic mapping shows that piRNAs are concentrated at a few loci (Brennecke et al., Cell, 128(6):1089-103 (2007)).

Primary transcripts for piRNAs are generated from the transposon regulatory regions of heterochromatin. These piRNAs are anti-sense, or complementary to transposon transcripts, and associated with both Piwi and Aub to trigger the amplification loop. Piwi/Aub cleaves target transposon transcripts between 10 and 11 nt from the 5′ end of anti-sense piRNA and subsequently generates Ago3-associated sense piRNA. Ago3 functions as another slicer, which recognizes the complementary sequence of piRNA cluster transcripts, and generates more Piwi/Aub-associated anti-sense strand piRNA. piRNAs are thought to function with Piwis endogenously to maintain transposon silencing.

### iv. External Guide Sequences (EGSs)

Ribonuclease P (RNase P) is a ribonucleoprotein complex found in all organisms. It is highly active in cells and is responsible for the maturation of 5′ termini of all tRNAs, which account for approximately 2% of total cellular RNA. Human RNase P has at least nine polypeptides and a RNA subunit (H1 RNA). One of the unique features of RNase P is its ability to recognize structures, rather than the sequences, of substrates. This allows RNase P to hydrolyze different natural substrates in vivo or in vitro. Accordingly, any complex of two RNA molecules that resembles a tRNA molecule can be recognized and cleaved by RNase P. One of the RNA molecules is called the external guide sequence (EGS). An mRNA sequence can be targeted for RNase P cleavage by using EGSs to hybridize with the target RNA and direct RNase P to the site of cleavage. The EGSs used to direct human RNase P for targeted cleavage resemble three-quarters of a tRNA molecule and consist of two sequence elements: a targeting sequence complementary to the mRNA sequence and a guide sequence, which is a portion of the natural tRNA sequence and is required for RNase P recognition.

An EGS is designed to base pair through hydrogen bonding mechanism with a target mRNA to form a molecular structure similar to that of a transfer RNA (tRNA). The EGS/mRNA target is then cleaved and inactivated by RNAse P. EGS are not consumed in this reaction, but instead can recycle as a catalyst through multiple cycles of target mRNA cleavage leading to target inactivation more effectively than conventional anti-sense DNA oligonucleotides. EGS combine the specificity of conventional antisense DNA for gene targeting with the catalytic potency of RNAse P. RNAse P is present in abundant quantities in all viable eukaryotic cells where it serves to process transfer RNA (tRNA) by cleavage of a precursor transcript.

Small RNA sequences have been described that target eukaryotic mRNA for degradation by endogenous RNAse P, a ubiquitous cellular enzyme that generates mature transfer RNA (tRNA) from precursor transcripts (Gopalan, et al., J Biol. Chem. 277:6759-6762 (2002); Guerrier-Takada and Altman, Methods Enzymol. 313:442-456 (2000); and Plehn-Dujowich and Altman, PNAS USA 95:7327-7332 (1998)). A small RNA termed an External Guide Sequence (EGS) can be designed that mimics certain structural features of a tRNA molecule when it forms a bimolecular complex with another RNA sequence contained within a cellular messenger RNA (mRNA). Thus, any mRNA can in principle be recognized as a substrate for RNAse P with both the EGS and RNAse P participating as cocatalysts although due to the complexity of the binding and cleavage steps the kinetics of the reaction are difficult to predict in vitro or in vivo (Gopalan, et al., J. Biol. Chem. 277:6759-6762 (2002) and Guerrier-Takada and Altman, Methods Enzymol. 313:442-456 (2000)).

Design of an EGS requires both knowledge of the mRNA primary sequence to be cleaved by RNAse P as well as the secondary structure of the mRNA sequences in the mRNA. EGS sequences must be complementary to the primary sequence of the targeted mRNA and the sequences in the mRNA must be exposed in a single-stranded conformation within the mRNA secondary structure in order to bind to the EGS. Secondary structure of target mRNA can be approximated by computer modeling or determined empirically using nucleases or other RNA cleaving reagents well known to one of ordinary skill in the art. This analysis may be useful in locating regions of mRNA for targeting with complementary oligonucleotides including conventional DNA antisense oligonucleotides and catalytic RNA.

RNAase P is a ribonucleoprotein having two components, an RNA component and a protein component. The RNA component of RNAase P is responsible for the catalytic cleavage which forms the mature 5′ ends of all transfer RNAs. RNAase P is endogenous to all living cells that have been examined. During the studies on recognition of substrate by RNAase P, it was found that E. coli RNAase P can cleave synthetic tRNA-related substrates that lack certain domains, specifically, the D, TψC and anticodon stems and loops, of the normal tRNA structure. For bacterial RNAse P a half-turn of an RNA helix and a 3′ proximal CCA sequence contain sufficient recognition elements to allow the reaction to proceed. Using these principles, any RNA sequence can be converted into a substrate for bacterial RNAase P by using an external guide sequence, having at its 5′ terminus nucleotides complementary to the nucleotides 3′ to the cleavage site in the RNA to be cleaved and at its 5′ terminus the nucleotides NCCA (N is any nucleotide).

EGS for promoting RNAase P-mediated cleavage of RNA has also been developed for use in eukaryotic systems as described by U.S. Pat. No. 5,624,824 to Yuan, et al., 6,610,478 to Takle, et al., WO93/22434 to Yale University, WO95/24489 to Yale University, and WO 96/21731 to Innovir Laboratories, Inc. As used herein, “external guide sequence” and “EGS” refer to any oligonucleotide or oligonucleotide analog that forms, in combination with a target RNA, a substrate for RNAase P.

An external guide sequence for promoting cleavage by RNAase P contains sequences which are complementary to the target RNA and which forms secondary and tertiary structures similar to portions of a tRNA molecule. In eukaryotes, including mammals, tRNAs are encoded by families of genes that are 73 to 150 base pairs long. tRNAs assume a secondary structure with four base paired stems known as the cloverleaf structure. The tRNA contains a stem, a D loop, a Variable loop, a TψC loop, and an anticodon loop. In one form, the EGS contains at least seven nucleotides which base pair with the target sequence 3′ to the intended cleavage site to form a structure like the stem, nucleotides which base pair to form stem and loop structures similar to the TψC loop, the Variable loop and the anticodon loop, followed by at least three nucleotides that base pair with the target sequence to form a structure like the D loop.

Preferred guide sequences for eukaryotic RNAase P consist of a sequence which, when in a complex with the target RNA molecule, forms a secondary structure resembling that of a IRNA cloverleaf or parts thereof. The desired secondary structure is determined using conventional Watson-Crick base pairing schemes to form a structure resembling a tRNA. Since RNAse P recognizes structures as opposed to sequences, the specific sequence of the hydrogen bonded regions is less critical than the desired structure to be formed. The EGS and the target RNA substrate should resemble a sufficient portion of the tRNA secondary and tertiary structure to result in cleavage of the target RNA by RNAase P. The sequence of the EGS can be derived from any tRNA. The sequences and structures of a large number of tRNAs are well known to one of ordinary skill in the art and can be found at least at http://rna.wustl.edu/tRNAdb/. The consensus sequence for RNAse P recognition of tRNA molecules is GNNNNNU. The sequence obtained from the stem of the tRNA is altered to be complementary to the identified target RNA sequence. Target RNA is mapped by techniques well known to one of ordinary skill in the art for the consensus sequence. Such techniques include digestion of the target mRNA with T1 nuclease. Digestion with T1 nuclease cleaves RNA after guanine (G) residues that are exposed in solution and single-stranded, but not after G residues that are buried in the RNA secondary structure or base paired into double-stranded regions. The reaction products form a ladder after size fractionantion by gel-electrophoresis. A T1 sensitive site is detected as a dark band is compared to the target mRNA sequence to identify RNAse P consensus sequences. The complimentary sequence from the target mRNA is used for the EGS. The complementary sequences may consist of as few as seven nucleotides, but preferably include eleven nucleotides, in two sections which base pair with the target sequence and which are preferably separated by two unpaired nucleotides in the target sequence, preferably UU, wherein the two sections are complementary to a sequence 3′ to the site targeted for cleavage.

The remaining portion of the guide sequence, which is required to cause RNAase P catalytic RNA to interact with the EGS/target RNA complex, is herein referred to as an RNAase P binding sequence. The anticodon loop and the Variable loop can be deleted and the sequence of the TAX loop can be changed without decreasing the usefulness of the guide sequence. External guide sequences can also be derived using in vitro evolution techniques (see U.S. Pat. No. 5,624,824 to Yuan, et al. and WO95/24489 to Yale University).

### v. Ribozymes and Other Catalytic RNAs

In another embodiment, the inhibitory RNA is a catalytic RNA, or a ribozyme. Ribozymes are described, for example, in PCT International Publication WO90/11364, published Oct. 4, 1990; Sarver et al, Science, 247:1222-1225 (1990). While ribozymes that cleave mRNA at site specific recognition sequences can be used to degrade target mRNAs the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature, 334:585-591 (1988). There are usually numerous potential hammerhead ribozyme cleavage sites within each nucleotide sequence of a target mRNA of known sequence. Preferably, the ribozyme is engineered so that the cleavage recognition site is located near the 5′ end of the target. mRNA. This functions to increase efficiency and minimize the intracellular accumulation of non-functional mRNA transcripts.

### B. Liposomes

Complexation of ODN with liposomes reduce enzymatic degradation of ODN in bladder and prevent formation of secondary and high-order solution structures and thereby reduce non-specific effects. Furthermore, the negative charge on the phosphate group of ODN inhibits their ability to pass through the mainly lipophilic cell membrane (Akira Murakami, Seitai-no-Kagaku, 49:309-314 (1998)). The methods to deliver ODN have been intensively investigated mainly aiming at improvement in penetration and transportation of oligonucleotides into cells. To solve such problems, the following methods were proposed in Yokoyama's review (Kazunari Yokoyama, Cellular Engineering, 16:1463-1473 (1997)): to conjugate ODN with cationically charged compounds such as poly-L-lysine and polyethyleneimine; to use transferrin/poly-L-lysine-conjugated DNAs in the presence of capsid of replication-defective adenovirus; to use fragments of the homeo domains of membrane-fused peptide derived from influenza virus HA surface protein (Bongartz, et al., Nucleic Acids Research, 22:4681-4688 (1994)) and Antennapedia protein of Drosophila; to bind oligonucleotides with folic acid or acialoglycoprotein receptor, or transferrin, so as to be targeted to specific cell surface receptors; to conjugate oligonucleotides with cholesterol; to encapsulate oligonucleotides into cationic lipids, and the like. These proposals mainly aim at improving penetration and transportation of oligonucleotide into cells, and in vitro experiments wherein oligonucleotides can be directly administered to cells, have provided good results. However, in vivo experiments have failed to show sufficient results, and safety was questionable.

Due to the inadequacies of these methods, there exists a need to safely improve stability and resistance to nuclease degradation as well as decrease toxicity and increase binding affinity to bladder surface of ODN. The high cost of gene-silencing (antisense ODN or siRNA therapeutics) is further evidence of the need to curb ODN degradation, prevent the formation of high-order structures and at the same time deliver sufficient amounts of ODN to the disease site. This problem is solved by utilizing site specific delivery of biocompatible liposome based delivery system.

Liposomes (LPs) are spherical vesicles, composed of concentric phospholipid bilayers separated by aqueous compartments. LPs have the characteristics of adhesion to and creating a molecular film on cellular surfaces. Liposomes are lipid vesicles composed of concentric phospholipid bilayers which enclose an aqueous interior (Gregoriadis, et al., Int J Pharm., 300:125-30 2005; Gregoriadis and Ryman, Biochem J., 124:58P (1971)). The lipid vesicles comprise either one or several aqueous compartments delineated by either one (unilamellar) or several (multilamellar) phospholipid bilayers (Sapra, et al., Curr Drug Deliv., 2:369-81 (2005)). The success of liposomes in the clinic has been attributed to the nontoxic nature of the lipids used in their formulation. Both the lipid bilayer and the aqueous interior core of liposomes can serve the purpose of treatment. Liposomes have been well studied as carrier of toxins for enhancing their efficacy at lower doses (Alam, et al., Mol Cell Biochem., 112:97-107 (1992); Chaim-Matyas, et al., Biotechnol Appl Biochem., 17 (Pt 1):31-6 (1993); de Paiva and Dolly, FEBS Lett., 277:171-4 (1990)); Freitas and Frezard, Toxicon., 35:91-100 (1997); Mandal and Lee, Biochim Biophys Acta, 1563:7-17 (2002)).

Liposomes have the ability to form a molecular film on cell and tissue surfaces and are currently being tested as possible therapeutic agents to promote wound healing and healing dry eye as a tear substitute. Clinical studies have proven the efficacy of liposomes as a topical healing agent (Dausch, et al., Klin Monatsbl Augenheilkd, 223:974-83 (2006); Lee, et al., Klin Monatsbl Augenheilkd, 221:825-36 (2004)). Liposomes have also been used in ophthalmology to ameliorate keratitis, corneal transplant rejection, uveitis, endophthalmitis, and proliferative vitreoretinopathy (Ebrahim, et al., 2005; Li, et al., 2007).

Liposomes have been widely studied as drug carriers for a variety of chemotherapeutic agents (approximately 25,000 scientific articles have been published on the subject) (Gregoriadis, N Engl J Med, 295:765-70 (1976); Gregoriadis, et al., Int J Pharm., 300:125-30 (2005)). Water-soluble anticancer substances such as doxorubicin can be protected inside the aqueous compartment(s) of liposomes delimited by the phospholipid bilayer(s), whereas fat-soluble substances such as amphotericin and capsaicin can be integrated into the phospholipid bilayer (Aboul-Fadi, Curr Med Chem., 12:2193-214 (2005); Tyagi, et al., J Urol., 171:483-9 (2004)). Topical and vitreous delivery of cyclosporine was drastically improved with liposomes (Lallemand, et al., Eur J Pharm Biopharm 56, 307-18 2003). Delivery of chemotherapeutic agents lead to improved pharmacokinetics and reduced toxicity profile (Gregoriadis, Trends Biotechnol., 13:527-37 (1995); Gregoriadis and Allison, FEBS Lett., 45:71-4 (1974); Sapra, et al., Curr Drug Deliv., 2:369-81 (2005)). More than ten liposomal and lipid-based formulations have been approved by regulatory authorities and many liposomal drugs are in preclinical development or in clinical trials (Barnes, Expert Opin Pharmacother., 7:607-15 (2006); Minko, et al., Anticancer Agents Med Chem., 6:537-52 (2006)). Fraser, et al. Urology, 2003; 61: 656-663 demonstrated that intravesical instillation of liposomes enhanced the barrier properties of dysfunctional urothelium and partially reversed the high micturition frequency in a rat model of hyperactive bladder induced by breaching the uroepithelium with protamine sulfate and thereafter irritating the bladder with KC1. Tyagi et al. J Urol., 171:483-489 (2004) reported that liposomes are a superior vehicle for the intravesical administration of capsaicin with less vehicle induced inflammation in comparison with 30% ethanol. The safety data with respect to acute, subchronic, and chronic toxicity of liposomes has been assimilated from the vast clinical experience of using liposomes in the clinic for thousands of patients. The safe use of liposomes for the intended clinical route is also supported by its widespread use as a vehicle for anticancer drugs in patients.

Liposomes are lipid vesicles composed of concentric phospholipid bilayers, which enclose an aqueous interior (Gregoriadis, et al., 2005; Gregoriadis and Ryman, 1971). The lipid vesicles comprise either one or several aqueous compartments delineated by either one (unilamellar) or several (multilamellar) phospholipid bilayers (Tyagi, et al., 2006). The lipid bilayer and the aqueous interior of liposomes have been used to deliver chemotherapeutic agents with improved pharmacokinetics and reduced toxicity (approximately 25,000 scientific articles have been published on the subject) (Gregoriadis, 1995; Gregoriadis and Allison, 1974; Sapra, et al., 2005).

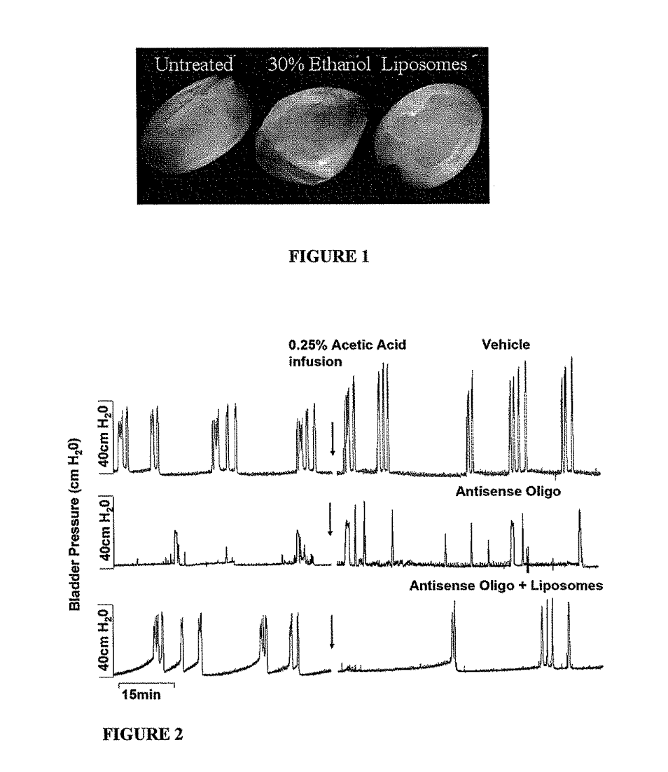

Effective antisense inhibition in cells requires efficient intracellular uptake and the delivery of antisense agents across impermeable cellular layer of bladder urothelium, which can be a particular problem for relatively large, hydrophilic molecules such as phosphothioate ODN to be used in proposed experiments (Tyagi, et al., 2006). Most of the delivery systems currently used are based on cationic lipids or cationic polymers. In fact, cationic liposome-ODN complexes have been able to successfully deliver ODN across different cells including bladder (Li, et al., 2008; Nogawa, et al., 2005). The development of cationic lipsomes has been helped by the easy and reversible complex formation between ODN and liposomes and the efficient delivery of their cargo into cells. Biophysical studies on interactions of liposomes with cells have suggested that liposomes can be adsorbed, or will fuse or transfer lipids with the cell membrane, and they can also be endocytosed inside the cell (Tyagi, et al., 2006). The success of liposomes in the clinic has been attributed to the nontoxic nature of the lipids and safety data has been assimilated from the vast clinical experience of using liposomes in the clinic in thousands of patients(Tyagi, et al., 2006) (FIG. 1).

### II. Methods of Manufacturing

Preparation of ODNs

Antagonists that reduce or inhibit expression of NGF, VEGF, TNF-alpha, or TGF-beta include inhibitory nucleic acids, including, but not limited to, ribozymes, triplex-forming oligonucleotides (TFOs), external guide sequences (EGSs) that promote cleavage by RNase P, peptide nucleic acids, antisense DNA, siRNA, and microRNA specific for nucleic acids encoding these proteins.

Useful inhibitory nucleic acids include those that reduce the expression of RNA encoding NGF, VEGF, TNF-alpha, or TGF-beta by at least 20%, 30%, 40%, 50%, 60%, 70%, 80%, 90% or 95% compared to controls. Expression of NGF, VEGF, TNF-alpha, or TGF-beta can be measured by methods well know to those of skill in the art, including northern blotting and quantitative polymerase chain reaction (PCR).

Inhibitory RNAs can be produced using methods known to those skilled in the art. They can be chemically synthesized, produced by in vitro transcription; expressed in cells from an expression plasmid or viral vector; or expressed in cells from a PCR-derived expression cassette. In vitro synthesis may be chemical or enzymatic, for example using cloned RNA polymerase (e.g., T3, T7, SP6) for transcription of the endogenous DNA (or cDNA) template, or a mixture of both. SiRNAs can also be produced by digestion of long dsRNA by an RNase III family enzyme (e.g., Dicer, RNase III). In a preferred embodiment, the inhibitory RNAs are obtained synthetically, for example, by chemically synthesizing a nucleic acid by any method of synthesis known to the skilled artisan. The synthesized inhibitory RNAs can then be purified by any method known in the art. Methods for chemical synthesis of nucleic acids include in vitro chemical synthesis using phosphotriester, phosphate or phosphoramidite chemistry and solid phase techniques, or via deoxynucleoside H-phosphonate intermediates (see U.S. Pat. No. 5,705,629 to Bhongle).

In vivo, inhibitory RNAs may be synthesized using recombinant techniques well known in the art (see e.g., Sambrook, et al., Molecular Cloning; A Laboratory Manual, Third Edition (2001). For example, bacterial cells can be transformed with an expression vector which comprises the DNA template from which the inhibitory RNAs are to be derived. The RNA can be purified by extraction with a solvent (such as phenol/chloroform) or resin, precipitation (for example in ethanol), electrophoresis, chromatography, or a combination thereof.

Methods for producing inhibitory RNAs that target mRNAs of known sequence are known in the art. One of skill in the art could readily produce inhibitory RNAs that downregulate the expression of any chosen mRNA in host using information that is publicly available. Inhibitory nucleic acids and methods of producing them are well known in the art. siRNA design software is available for example at http://i.cs.hku.hk/˜sirna/software/sirna.php. Synthesis of nucleic acids is well known see for example Molecular Cloning: A Laboratory Manual (Sambrook and Russel eds. 3rd ed.) Cold Spring Harbor, N.Y. (2001).

Methods for producing siRNA are known in the art. Because the nucleotide sequences that encode NGF, VEGF, TNF-alpha, or TGF-beta are known, one of skill in the art could readily produce siRNAs that downregulate NGF, VEGF, TNF-alpha, or TGF-beta expression in a host using the information that is publicly available.

Manufacturing of Liposomes:

Methods of manufacturing of the liposomes are described in the literature cited above and are well known.

In one embodiment, aqueous liposome suspensions are produced by microfluidization. The end product may be subject to a series of stability problems such as aggregation, fusion and phospholipid hydrolysis (Nounou, et al., Acta Poi Pharm., 62:381-91 (2005)).

The liposomal product must possess adequate chemical and physical stability before its clinical benefit can be realized (Torchilin, Adv Drug Deliv Rev., 58:1532-55 (2006)). In a preferred embodiment, dehydrated liposomes are formed from a homogenous dispersion of phospholipid in a tert-butyl alcohol (TBA)/water cosolvent system. The isotropic monophasic solution of liposomes is freeze dried to generate dehydrated liposomal powder in a sterile vial. The freeze drying step leaves empty lipid vesicles or dehydrated liposomes after removing both water and TBA from the vial. On addition of physiological saline, the lyophilized product spontaneously forms a homogenous liposome preparation (Amselem, et al., J Pharm Sci., 79:1045-52 (1990); Ozturk, et al., Adv Exp Med Biol., 553:231-42 (2004)). Low lipid concentrations works ideally for this method because lipid and TBA ratio is the key factor affecting the size and the polydispersity of resulting liposome preparation.

Preparation of Liposomal ODN:

In a preferred embodiment, Liposomal ODN is prepared by a dehydration-rehydration method with slight modifications. Liposomes prepared in the previous step are hydrated with a solution of ODN in water for injection (50 units/ml) at 25° C. Then the mixture is incubated for 30-120 min at the temperature of 25° C. using water bath to form oligolamellar hydration liposomes. Mannitol is added to the final mixture at a concentration of 0.5%, 1%, 2.5% and 5% mannitol (w/v), respectively before freezing in acetone-dry ice bath. Mannitol acts as a cryoprotectant in the freeze-drying process. The frozen mixture is lyophilized at −40° C. and 5 milibar overnight. The lyophilized cake is resuspended with saline to the desired final concentration of ODN. The free ODN is removed from entrapped ODN by centrifugation at 12,000×g for 30 min using ultracentrifuge. After washing three times, the precipitates are again resuspended in saline.

In the preferred embodiment the liposome to oligonucleotide molar ratio is at least about 1:1 to about 20:1.

### III. Administration of Liposomal ODN

Diseases of the LUT are often localized in bladder. As a result, the ODN can be placed in direct contact with bladder tissue through simple intravesical administration. Further, the response of the treatment can be easily determined with cystoscopy and urine cytokine analysis. One of the major obstacles to successful bladder uptake of ODNs is the presence of a glycosaminoglycan layer on transitional cell epithelia, which may act as a significant barrier (Ruponen, et al. Biochim. Biophys. Acta, 1415: 331-41 (1999)).

The method for treating a LUT symptoms in a subject can include administering to the subject an ODN antagonist of NGF, VEGF, TNF-alpha, or TGF-beta on a regular basis to treat disease in the subject.

The most preferred embodiment is described in the examples, wherein the method includes (1) administering an about 10 to 30 mer oligonucleotide comprising a base sequence complementary to a m-RNA or a m-RNA precursor of genes, of which expression is to be controlled or inhibited;(2) allowing the oligonucleotide to form double strand together with the m-RNA or m-RNA precursor in cells; and(3) inhibiting the translation of m-RNA by ribosome, or inhibiting the cleavage of double strand by RNase H, or the splicing of m-RNA precursor; thereby suppressing gene expressions.

Methods of instillation are known. See, for example, Lawrencia, et al., Gene Ther., 8:760-8 (2001); Nogawa, et al., J Clin Invest., 115:978-85 (2005); Ng, et al., Methods Enzymol., 391:304-13 (2005); Tyagi, et al., J Ural., 171:483-9 (2004); Trevisani, et al., J Pharmacol Exp Ther., 309:1167-73 (2004); Trevisani, et al., Nat Neurosci., 5:546-51 (2002)); (Segal, et al., 1975). (Dyson, et al., 2005). (Batista, et al., 2005; Dyson, et al., 2005). The volume of liposome-ODN is important in the efficacy of delivery. Routine experimentation can be used to optimize the delivery volume.

The disclosed liposomes can also be used to instill therapeutic agents to other sites such as the urinary tract including the urethra, bladder, ureter and intrarenal collecting system; gynecological sites such as vaginal, uterus, fallopian tube; gastrointestinal sites including mouth, esophagus, stomach, intestine, colon, rectum, anus; and the outer or inner ear; skin, nose.

Liposome encapsulation solves the problems with poor absorption after instillation. Since ODN is entrapped inside the liposomes, it is not vulnerable to dilution by urine and localized concentration of ODN at liposome surface is high enough to hasten the passive diffusion of leached ODN from liposomes adherent on the bladder surface. The lipid barrier of liposomes can also prevent the access of nucleases in urine from cleaving the ODN before it is absorbed by the bladder.

The present invention will be further understood by reference to the following non-limiting examples.

### EXAMPLE 1: BLADDER UPTAKE OF ANTISENSE ODN

### Materials and Methods

18mer fluorescent ODN were custom made by Integrated DNA technologies (San Diego, Calif.). Cationic liposomes were composed of DOTAP (N-[1-(2,3-Dioleoyloxy)propyl]-N,N,N-trimethylammonium Methylsulfate) and were made by thin film hydration method and hydrated with nuclease free water with the final lipid concentration of 7mM. Fluorescent ODN with the sequence 5′GCCCGAGACGCCTCCCGA3′ (SEQ ID NO: 1) were dispersed in nuclease free water at a concentration of 6 μM and were complexed with liposomes by incubation at room temperature for 30 min. Molar ratio of ODN to lipid in the liposomal complex was 1:10.

Bladder uptake studies: Female Sprague—dawley rats were anaesthetized with isoflurane and the bladders were catheterized 24-gauge angiocatheters (Becton Dickinson), washed with saline to instill 0.5 ml of either saline or liposomal fluorescent ODN (6 μM), followed by a purse-string suture placed around the urethra to occlude for 30 min. The string around the urethra was removed at the end of 30 min and the rats restarted their spontaneous voiding in metabolic cages at the end of instillation.

Rats were sacrificed at 8 and 24 h after instillation. Bladders were harvested at the time of sacrifice and cryopreserved for cryosectioning into tissue sections of 8 microns. Sections were examined for fluorescence signal from instilled ODN in bladder by Zeiss LSM 510 META confocal microscope attached to a CCD camera. The uptake of ODN was monitored using the bright red fluorescence.

Cystometric studies: The efficacy of antisense treatments was assessed 24 h after instillation by saline and acetic acid cystometry under urethane anesthesia (1.0 g/kg subcutaneously). Their body temperature was maintained in the physiologic range using a heating lamp. A transurethral catheter (PE-50) connected by a three-way stopcock to a pressure transducer and to a syringe pump was used to record intravesical pressure and to infuse solutions into the bladder. A control cystometrogram (CMG) was performed by slowly filling the bladder with saline (0.04 mL/min) to elicit repetitive voiding followed by 0.25% acetic acid (AA) infusion to induce bladder irritation. The intercontractile interval (ICI) of the reflex bladder contractions during saline and AA was recorded.

Immunohistochemistry: Bladders at the end of cystometry were cryopreserved and 8 μm thick cryosections were washed in PBS and preincubated with PBS containing 20% normal serum (Jackson Immunoresearch) and 0.2% Triton X-100 (VWR International,) for 2 h at room temperature. The primary polyclonal rabbit H-20 antibody (1:50) (Santa Cruz Biotechnology, Santa Cruz, Calif.) for NGF was applied in PBS containing 5% normal serum, 0.2% Triton X-100 for 16-18 h at 4° C. Sections were washed in PBS containing 0.1% BSA, 0.1% Triton X-100, 4× for 5 min each at room temperature. The secondary donkey anti-rabbit Alexa Fluor 488 (1:200) (Molecular Probes, Eugene, Oreg.) antibody was applied for 2 h at room temperature in PBS containing 0.1% BSA, 0.1% Triton X-100. Washing was performed 3× at room temperature in PBS, and sections were mounted with an aqueous mounting medium. Immunostaining was analyzed with a Nikon confocal microscope. Rat bladders harvested after instillation were cryosectioned for viewing under confocal microscope. The uptake of ODN after instillation was monitored using the bright red fluorescence of TYE 563 in the 8 micron thick sections.

### Results

Bladder Uptake: Bladder uptake accumulation was best seen at 24 h , while intensity of fluorescence was greater at 8h. The fluorescence of ODN is not surface bound to bladder lumen, it is intracellular at 8 h and at 24 h. Surface bound fluorescent ODN will be washed away by over dozen voiding episodes of normal rat in awake condition. Localization of fluorescence in urothelium demonstrates successful bladder uptake and retention in target cells presumably due to binding with target mRNA. The fluorescence uptake studies demonstrate influx of liposomal antisense ODN from single instillation.

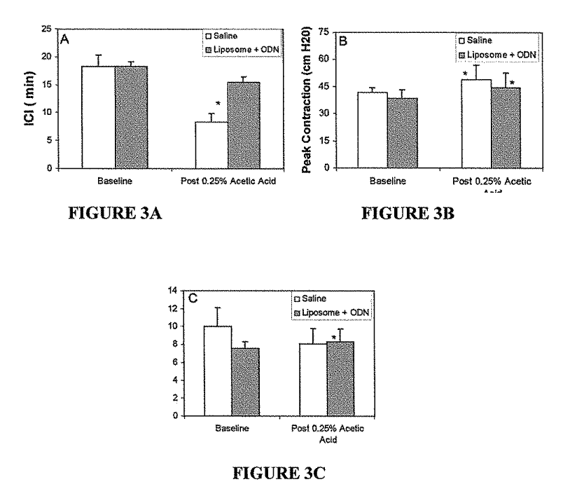

Cystometry: Baseline CMG under saline infusion was indistinct between the groups and instillation of antisense ODN had no effect on baseline CMG as evident from mean ICI of 18.28±2.06min in the vehicle treated group and 18.29±1.62 min in the antisense treated group (n=6). The acetic acid induced bladder overactivity was noticed in vehicle-treated rats as evident from mean percent reduction of (49.71±9.68%) in ICI from baseline values (FIG. 2, top tracing). Pretreatment of antisense ODN encapsulated in liposomes blocked the acetic acid induced irritation mediated by NGF (FIG. 2, bottom tracing) with only 14.17±3.71% reduction in mean ICI from baseline (n=6). The difference in the percent reduction in the two treatment groups was statistically significant using unpaired student's t test (p<0.05) (FIG. 3A). The need for liposomes in successful delivery of antisense oligo targeting NGF is demonstrated by the acetic acid induced bladder overactivity was noticed in the rats instilled with oligos alone without liposomes (FIG. 2, middle tracing). Analysis of other cystometric parameters revealed significant rise in peak micturition pressure following acetic acid infusion relative to saline infusion at baseline (FIG. 3B). There was no change in pressure threshold for each contraction (FIG. 3C) either at baseline or after acetic acid infusion in either treatment groups. Delivery of NGF antisense oligos probably blunts the rapid rise of NGF that occurs upon acetic acid irritation.

NGF immunostaining: The immunoreactivity for NOF was noticed in the detrusor region of all sections and it was absent in the apical urothelial cells of rats left untreated with AA. The NGF immunoreactivity appeared to increase several fold in detrusor and its presence was distinctly noticed in apical urothelial umbrella cells following infusion of AA in vehicle treated rat bladder. The AA-induced NGF immunoreactivity in apical urothelial umbrella cells was also noticed in rats instilled with naked ODN without liposomes. In contrast, the NGF immunoreactivity in urothelium including a subpopulation of intermediate-to-apical urothelial cells was reduced to levels observed in control rats by pretreatment with liposomal ODN. Control sections incubated in the absence of primary or secondary antibody were also processed and evaluated for specificity or background staining levels. In the absence of primary antibody, no positive immunostaining was observed.

### Discussion

Although great progress has been made recently in translating antisense research into clinical therapies in oncology and in ophthalmology, applied research for bladder diseases has lagged behind. The primary impediment in the development of antisense and siRNA therapeutics is inefficient extracellular delivery and uptake of the oligonucleotides (Tyagi, et al., Mol. Pharm, 3(4):398-406 (2006) and Tyagi, et al., Mol. Pharm, 3:369-79 (2006)). The uptake of ODN without liposomes is believed to be limited by the glycosaminoglycan layer of the inner bladder surface as supported by cystometric and immunohistochemical data discussed above. Additionally, anionic charge, hydrophilicity and the inherent size of the ODN may inhibit the intracellular passage through the cellular lipid bilayer (Tyagi, et al., Mol. Pharm, 3(4):398-406 (2006) and Tyagi, et al., Mol. Pharm, 3:369-79 (2006)). The CMG data presented above support the hypothesis that NGF is a chemical mediator responsible for changes in urinary bladder function by either acting directly on sensory nerve endings or by indirect increase in the expression of sensory neuropeptides, such as substance P and CGRP, neuropeptides that are known to initiate local neuroinflammatory responses and enhance sensory neuron excitability. As the results above show, successful delivery of antisense targeting NGF blunts the rapid rise of NGF following acetic acid irritation is supported by the immunostaining of NGF in urothelium noticed in vehicle and naked ODN. The CMG and immunostaining data taken together support the fact that rise in NGF content in bladder following acetic acid irritation, can be blocked at the translation step.

### EXAMPLE 2. EFFECT OF INTRAVESICAL NGF ANTISENSE TREATMENT ON CYP INDUCED DO

### Materials and Methods

The in vivo efficacy of NGF antisense PNA-TAT conjugates instilled intravesically was examined in DO by Cyclophosphamide CYP (dose 100 mg/kg i.p.) to demonstrate the importance of effective bladder delivery of antisense therapeutics. Cystometry was performed in an awake or urethane anesthetized condition. First, under isoflurane anesthesia, the bladder was exposed with a lower midline abdominal incision, and PE-50 tubing with a cuff at the end was inserted into the bladder through the bladder dome. After the surgery, rats were placed in straining cages and allowed to recover from isoflurane anesthesia for 1-2 hours to obtain cystometry in a conscious condition, or anesthetized with urethane (1.2 g/kg, s.c.). The number and amplitude of non-voiding bladder contractions prior to voiding were monitored to evaluated DO induced by SCI.

### Results

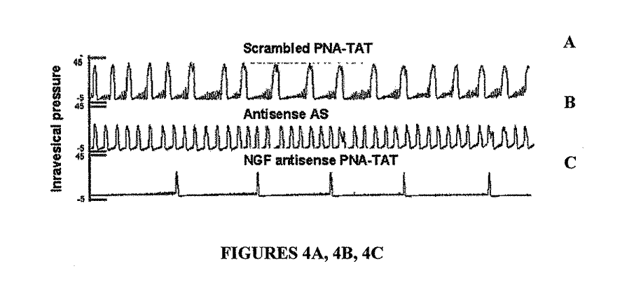

The results demonstrate the in vivo utility of antisense ODN with the different backbone of peptide nucleic acid conjugated with cell penetrating peptide, TAT. The results show that the delivery vehicle is essential for bladder uptake of nucleic acids as naked peptide nucleic acid (PNA) was unable to cross inside the bladder cells but PNA conjugated with tethering TAT peptide (PNA-TAT) can be delivered into urothelial cells when locally instilled into the bladder. The cell penetrating cationic peptide served the same function as cationic liposomes did for ODN delivery in bladder. Pre-treatment of NGF antisense PNA-TAT blocked the CYP induced NGF overexpression and suppressed the subsequent NGF mediated DO as evident from cystometry (FIGS. 4A, 4B and 4C). Cystometry revealed CYP (100 mg/kg) induced bladder overactivity in rats treated with scrambled sequence (Scrambled PNA-TAT, top tracing, FIG. 4A) and in aqueous solution of native antisense ODN (Antisense AS, middle tracing, FIG. 4B). The CYP-induced bladder overactivity was only suppressed in rats instilled with NGF antisense PNA-TAT (bottom tracing, FIG. 4C), demonstrating the importance of effective bladder delivery of antisense therapeutics.

Modifications and variations of the methods and materials described herein will be apparent to those skilled in the art and are intended to come within the scope of the following claims. All references cited herein are specifically incorporated by reference.

We claim:

1. A method of inhibiting expression of a target mRNA in bladder comprising administering an effective amount of inhibitory oligonucleotide molecule in lipid vesicles by intravesicular instillation.

2. The method of claim 1 wherein at least one symptom of a disease is mediated at least in part by the presence of abnormal mRNA in the bladder to be treated.

3. The method of claim 1 wherein the oligonucleotide is selected from the group consisting of dsRNA, siRNA, shRNA, miRNA, piRNA, external guide sequences, ribozymes, and other short catalytic RNAs.

4. The method of claim 3 wherein the oligonucleotide is an antisense ODN, less than 30 nucleotides in length.

5. The method of claim 1 wherein the lipid vesicle is a liposome composed of cationic and non-ionic lipids.

6. The method of claim 1, comprising lipid vesicle and oligonucleotide wherein the liposome to oligonucleotide molar ratio is at least about 1:1 to about 20:1.

7. The method of claim 3, in which the oligonucleotide has a base sequence substantially complementary to a sequence of a target mRNA or of a target mRNA precursor.

8. The method of claim 1, wherein the target mRNA is complementary to a gene encoding a protein selected from the group consisting of nerve growth factor, vascular endothelial growth factor, tumor necrosis factor-alpha, and tumor necrosis factor-beta.

9. The method of claim 1, wherein the oligodeoxynucleotide comprises a phosphorodiester backbone, a phosphorothioate backbone, locked nucleic acid, peptide nucleic acid, tricyclo-DNA, decoy oligonucleotide, ribozymes, spiegelmers, and CpG oligomers.

10. The method of claim 1, wherein the target mRNA is instilled into the genitourinary tract including the kidney, ureter, urethra, prostate and vagina.

11. The method of claim 1, wherein the target mRNA is instilled into the hollow cavities of the body including the oral nasal cavity, ear, respiratory tract, anus, rectum and digestive track.

12. A formulation for use in the method of claim 1.


SEQUENCE LISTING (1 sequence)

SEQ ID NO: 1 - DNA, 18 bp, Artificial Sequence
  Synthetic antisense oligonucleotide
gcccgagacgcctcccga